(19) United States
(12) Patent Application Publication
(10) Pub. No.: US 2009/0011428 A1
(43) Pub. Date: Jan. 8, 2009

(54) Fluid Membrane-Based Ligand Display System for Live Cell Assays and Disease Diagnosis Applications

(75) Inventors: Jwa-Min Nam, Seoul (KR); Pradeep M. Nair, Berkeley, CA (US); Richard M. Neve, San Mateo, CA (US); Joe W. Gray, San Francisco, CA (US); John T. Groves, Berkeley, CA (US)

Correspondence Address: LAWRENCE BERKELEY NATIONAL LABORATORY, ONE CYCLOTRON ROAD, MAIL STOP 90B, UNIVERSITY OF CALIFORNIA, BERKELEY, CA, 94720, US

(73) Assignee: The Regents of the University of California, Oakland, CA (US)

(21) Appl. No.: 12/161,553

(22) PCT Filed: Jan. 18, 2007
(86) PCT No.: PCT/US07/60721

§ 371 Date: Jul. 21, 2008

Related U.S. Application Data
(60) Provisional application No. 60/760,258, filed on Jan. 18, 2006.

(51) Int. Cl. G01N 33/53

(52) U.S. Cl. 435/7.1

(57) ABSTRACT

A supported membrane based, strategy for the presentation of soluble signaling molecules to living cells is described. In this system, the fluidity of the supported membrane enables localized enrichment of ligand density in a configuration reflecting cognate receptor distribution on the cell surface. Display of a ligand in non-fluid supported membranes produces significantly less cell adhesion and spreading, thus demonstrating that this technique provides a means to control functional soluble ligand exposure in a surface array format. Furthermore, this technique can be applied to tether natively membrane-bound signaling molecules such as ephrin A1 to a supported lipid bilayer. Such a surface can modulate the spreading behavior of metastatic human breast cancer cells displaying ligands and biomolecules of choice. The SLB microenvironment provides a versatile platform that can be tailored to controllably and functionally present a multitude of cell signaling events in a parallel surface array format.

### CROSS-REFERENCE TO RELATED APPLICATIONS

This application claims priority to U.S. Provisional Patent Application No. 60/760,258, filed on Jan. 18, 2006, which is hereby incorporated by reference in its entirety.

### STATEMENT OF GOVERNMENTAL SUPPORT

This work was supported by the Department of Energy under Contract No. DE-AC02-05CH11231. The government has certain rights in the invention.

### BACKGROUND OF THE INVENTION

1. Field of the Invention

The present invention relates to field of ligand display in a surface assay format that allows for systematic, patterned presentation of soluble ligands to live cells, specifically to the field of supported membranes for the presentation of soluble signaling molecules to living cells. The present invention also relates to surface display of molecules for high-throughput functional genetic studies and screening therapeutic agents.

2. Related Art

Cell communication modulates numerous biological processes including proliferation, apoptosis, motility, invasion and differentiation. Correspondingly, there has been significant interest in the development of surface display strategies for the presentation of signaling molecules to living cells. This effort has primarily focused on naturally surface-bound ligands, such as extracellular matrix components and cell membranes. Soluble ligands (e.g. growth factors and cytokines) play an important role in intercellular communications, and their display in a surface-bound format would be of great utility in the design of array-based live cell assays. Recently, several cell microarray systems that display cDNA, RNAi, or small molecules in a surface array format were proven to be useful in accelerating high-throughput functional genetic studies and screening therapeutic agents. See methods described in J. Ziauddin, D. M. Sabatini, Nature 2001, 411, 107; D. B. Wheeler, S. N. Bailey, D. A. Guertin, A. E. Carpenter, C. O. Higgins, D. M. Sabatini, Nat. Methods 2004, 1, 127; and S. N. Bailey, D. M. Sabatini, B. R. Stockwell, Proc. Natl. Acad. Sci. U.S.A. 2004, 101, 16144. These surface display methods provide a flexible platform for the systematic, combinatorial investigation of genes and small molecules affecting cellular processes and phenotypes of interest. In an analogous sense, it would be an important advance if one could display soluble signaling ligands in a surface assay format that allows for systematic, patterned presentation of soluble ligands to live cells. Such a technique would make it possible to examine cellular phenotypes of interest in a parallel format with soluble signaling ligands as one of the display parameters.

A surface detector array using a fluid membrane on a substrate is described in U.S. Pat. No. 6,228,326, and co-pending U.S. patent application Ser. No. 10/076,727, describes the modulation of cellular adhesion onto fluid lipid membranes that are displayed on substrates, both of which are hereby incorporated by reference.

### SUMMARY OF THE INVENTION

The present invention provides for a ligand-modified fluid supported lipid bilayer (SLB) assay system that can be used to functionally display soluble ligands to cells in situ. Ligand-modified fluid supported lipid bilayer (SLB) assay system. Soluble ligands are displayed on a SLB surface, combining both solution behavior (the ability to become locally enriched by reaction-diffusion processes) and solid behavior (the ability to control the spatial location of the ligands in an open system) in a single system.

Thus the invention provides a ligand-modified fluid supported lipid bilayer (SLB) assay system to functionally display soluble ligands to cells in situ, the SLB assay system comprising a substrate supporting a membrane bilayer having an aqueous layer between the substrate and the bilayer, wherein a soluble signaling ligand is displayed by the membrane bilayer thereby permitting a cell to interact with the signaling ligand. A thin aqueous layer is between the bilayer and the substrate.

The lipid bilayer displays a biological molecule, wherein the biological molecule is an affinity tag having a known binding partner or having a known affinity molecule that can be attached. In one embodiment, the biological molecule displayed by the lipid bilayer is biotin, thereby permitting a binding pair of streptavidin and biotin to be used. In another embodiment, the biological molecule displayed is a suitable affinity tag selected from the group consisting of: polysaccharides, lectins, selecting, nucleic acids (both monomeric and oligomeric), proteins, enzymes, lipids, antibodies, and small molecules such as sugars, peptides, aptamers, drugs, and other ligands, and thereby forming a bilayer displaying the affinity tag.

A labeled ligand-chimera is captured by the affinity tag and thereby displayed by the lipid bilayer. In one embodiment, the labeled ligand-chimera is an epidermal growth factor (EGF) protein attached to streptavidin and a detectable label. In another embodiment, the ligand of the labeled ligand-chimera is a soluble signaling ligand attached to the binding pair of the displayed biological molecule and a detectable label. In a preferred embodiment, the detectable label is a fluorescent molecule.

In one embodiment, the ligand of the labeled ligand-chimera is an ephrin A1 (EA1) protein attached to an affinity tag with a known binding partner and a detectable label. In another embodiment, the ligand of the labeled ligand-chimera is a glycosylphosphatidyl inositol (GPI) anchored signaling ligand attached to both an affinity tag with a known binding partner and a detectable label. And in another embodiment, the ligand of the labeled ligand-chimera is a membrane-anchored signaling ligand attached to both an affinity tag with a known binding partner and a detectable label.

The invention further provides a method of making an assay system comprising the steps of: (a) providing a substrate having a thin aqueous layer; (b) condensing a vesicle displaying an affinity tag by vesicle fusion process onto the thin aqueous layer, whereby a supported bilayer displaying the affinity tag is produced; (c) providing a labeled ligand-chimera which also displays a ligand that binds to the affinity tag displayed on the supported bilayer; (d) contacting and binding the labeled ligand-chimera with the affinity tag displayed on the supported bilayer. The method further comprising a step (e) contacting a live cell with the labeled ligand-chimera bound to the affinity tag displayed on the supported bilayer to observe cell-cell interactions.

The present invention benefits from the naturally fluid state of the supported membrane, which allows surface-linked ligands to diffuse freely in two dimensions. Ligands can become reorganized beneath cells, by reaction-diffusion processes, and may also adopt spatial configurations reflecting those of their cognate receptors on the cell surface. Using a supported bilayer system as described herein resulted in marked differences in the response of cells to membrane surface displayed soluble ligands as a function of membrane fluidity. Tethering of soluble signaling molecules to fluid supported membranes further provides opportunities to use membrane fabrication technologies to present soluble components within a surface array format.

### BRIEF DESCRIPTION OF THE DRAWINGS

FIG. 1A is a conceptual schematic of the fluid membrane-based soluble ligand display strategy. FIG. 1B is a schematic showing the fluid membrane-tethered EGF-based cell assay and fluorescence recovery after photobleaching (FRAP) experiments to test the fluidity of both EGF molecules and lipids on a cover glass slide (Tnset; false colors were used for fluorescence images). *Attofluor cell chamber was used throughout the addition of EGF to the SLB, FRAP experiments, the addition and incubation of cells on the SLB, and imaging processes. FIG. 1C is a larger view of the assay system.

FIG. 2 is a panel of bright field images of cells on supported lipid bilayers.

FIG. 3 is a set of images showing cell attachment to the EGF-modified SLB and EGF cluster formation. A. Bright field and fluorescence images were recorded over incubation time and fluorescence images show dynamic clustering of EGF within a cell. B. Bright field (left) and fluorescence (right) images of a cell on the EGF-modified SLB after 20 hr incubation at 37° C.

FIG. 4 is a set of images showing cells cultured at 37° C. for 20 hrs on fluid (DMOPC, top panels) and non-fluid (DPPC, bottom panels) EGF-SLB surfaces.

FIG. 5 is a schematic showing a metastatic cancer cell and its release mechanism (A) and supported membrane-based EphA2 array for metastasis study (B).

FIG. 6 presents the analysis of breast cancer cell line collection using the SLB system. (A) Western blot analysis of EphA2 and Erb3 in breast cancer cell lines. (B) Luminal and basal clusters in Affymetrix expression array analysis. (C) 3D cultures of breast cancer cell lines showing increased invasiveness of EphA2-expressing cells. (D) Western analysis of MCF10a cultures showing reciprocal EphA2/ErbB3 expression under different growth conditions.

FIG. 7 is a diagram of a hybrid live T cell-supported membrane junction. Receptors on the cell surface engage cognate ligands in the supported membrane and become subject to constraints on mobility imposed by physical barriers. The cytoskeleton is represented schematically to reflect the active source of central organization observed in our experiments.

FIG. 8 is a panel of photographs showing synapse formation is altered by geometrical constraints of the substrate in the SLB system. T cells were incubated with fluorescently labeled anti-TCR H57 Fab (green) before being introduced to supported bilayers containing GPI-linked pMHC (unlabeled) and ICAM-1 (red). Chromium lines are visible in brightfield, although they are only 100 nm across, verified by electron microscopy. Images are at 10 min after cells were introduced. IS on unpatterned substrate (A), 2-mm parallel lines (B), 5-mm square grid (C), and concentric hexagonal barriers (spacing 1 mm) (D). TCR distribution (grayscale) on 1-mm square grid (E). Transport map of (E) formed by drawing arrows toward the TCR cluster within the enhanced grid (F).

FIG. 9 is a set of photographs showing TCR-specific phosphotyrosine (pY) signaling in native and repatterned synapses cultured on the SLB system. T cells, which had been incubated with fluorescently labeled anti-TCR H57 Fab, were allowed to interact with pMHC-ICAM membranes for either 2 or 5 min before being fixed and stained for pY. (A) Synapse on unpatterned membrane at 2 min. TCR clusters are distributed, and relatively enhanced pY staining colocalizes with each cluster. The diffuse ring of pY staining in the periphery is likely associated with cortical actin. (B) Synapse on a 2-mm chromium grid at 2 min. (C) Synapse on unpatterned membrane at 5 min. (D) Synapse on a 2-mm chromium grid at 5 min. (E) Statistical results for % TCR colocalization with pY. Black, cells off pattern; gray, cells on 2-mm grids. Results are from three independent experiments at 2 min (a minimum of 9 cells per experiment both on and off patterns; total 31 on, 51 off) and four independent experiments at 5 minutes (a minimum of 7 cells per experiment on and off patterns; total 39 on, 53 off). Data from the 1-min time point (not shown) had extremely high standard deviation because cell population was not well synchronized. (F) Intracellular calcium is elevated in cells on grids. T cells were loaded with the ratiometric calcium-sensitive dye fura-2 and allowed to interact with pMHC-ICAM membranes. Fura-2 fluorescence emission ratio was integrated from 5 min to 20 min in cells on and off 2-mm grids (five independent experiments; total 49 on, 57 off).

FIG. 10 is a set of bright field images of metastatic human breast cancer cells (MDAMB231) cultured on Ephrin A1-functionalized supported lipid bilayer (EA1-SLB) (A) and ephrin-free supported lipid bilayer (SLB). (B) Graphs showing percent of total MDAMB231 cells spread on EA1-SLB and SLB, and (C) showing the number of adhered MDAMB231 cells/mm² on EA1-SLB and SLB. (D) Data was collected from multiple 0.92 mm² areas of a single EA1-SLB substrate and a single SLB substrate.

FIG. 11 is a set of bright field images of non-metastatic human breast cancer cells (T47D) cultured on Ephrin A1-functionalized supported lipid bilayer (EA1-SLB) (A) and ephrin-free supported lipid bilayer (B).

### DETAILED DESCRIPTION OF THE PREFERRED EMBODIMENT

In a preferred embodiment, a ligand-modified fluid supported lipid bilayer (SLB) assay system as herein described is used to functionally display soluble ligands to cells in situ. In a preferred embodiment, the SLB assay system is comprised of a substrate supporting a membrane bilayer having an aqueous layer between the substrate and the bilayer, wherein a soluble signaling ligand is displayed by the membrane bilayer thereby permitting a cell to interact with the signaling ligand. By displaying soluble ligands on a SLB surface, both solution behavior (the ability to become locally enriched by reaction-diffusion processes) and solid behavior (the ability to control the spatial location of the ligands in an open system) could be combined.

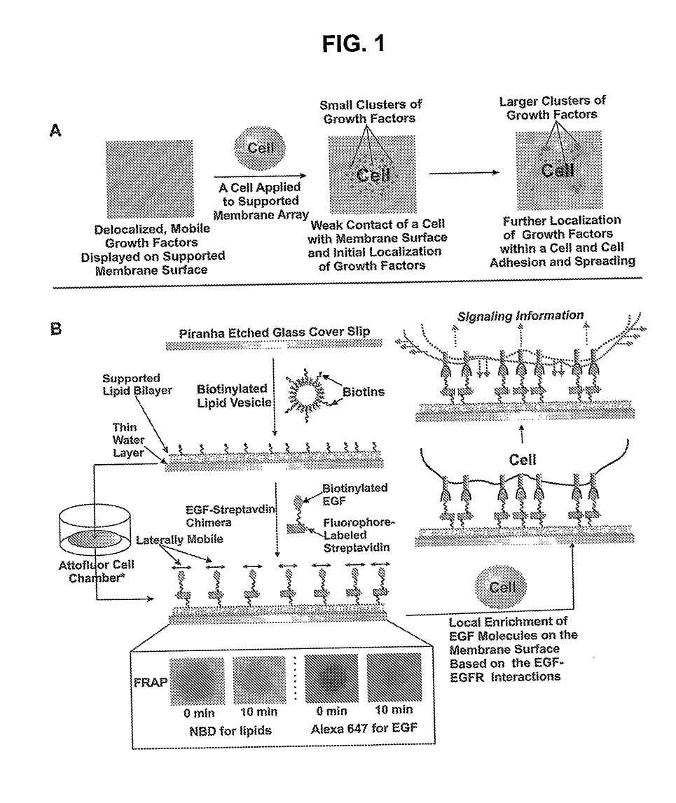

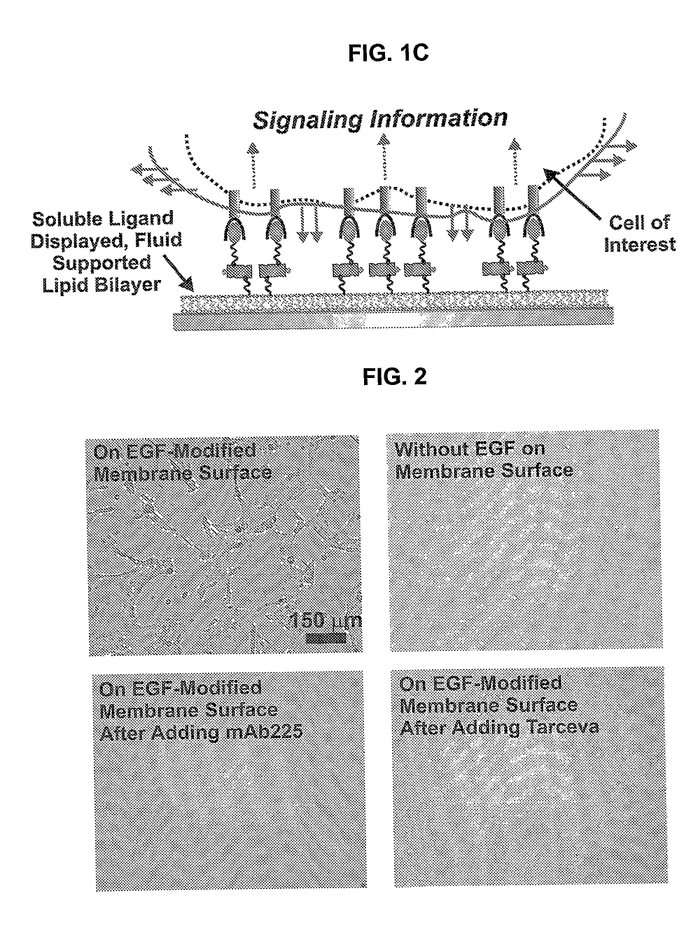

In a preferred embodiment, the assay system uses the naturally fluid state of the supported membrane, which allows surface-linked ligands to diffuse freely in two dimensions. Ligands can become reorganized beneath cells, by reaction-diffusion processes, and may also adopt spatial configurations reflecting those of their cognate receptors on the cell surface (FIG. 1A). This provides a significant benefit over conventional cell signaling and culturing systems that present inflexible distributions of signaling molecules. In this study described in the Examples, marked differences were observed in the response of cells to membrane surface displayed soluble ligands as a function of membrane fluidity. Tethering of soluble signaling molecules to fluid supported membranes provides opportunities to use membrane fabrication technologiesto display soluble components within a surface array format. Such membrane fabrication technologies may include those described by J. T. Groves, L. K Malial, C. R. Bertozzi, Langmuir 2001, 17, 5129; J. T. Groves, M. L. Dustin, J. Immunol. Meth. 2003, 278, 19; E. Sackmann, M. Tanaka, Trends Biotechnol. 2000, 18, 58; J. T. Groves, Angew. Chem. Int. Ed. 2005, 44, 3524; C. K. Yee, M. L. Amweg, A. N. Parikh, J. Am. Chem. Soc. 2004, 126, 13962; M. A. Holden, S.-Y. Jung, T. Yang, E. T. Castellana, P. S. Cremer, J. Am. Chem. Soc. 2004, 126, 6512; and L. Kam, S. G. Boxer, Langmuir 2003, 19, 1624, all of which are hereby incorporated by reference.

In another embodiment, a method of making the assay system is provided comprising the steps of: (a) providing a substrate having a thin aqueous layer; (b) condensing a vesicle displaying an affinity tag by vesicle fusion process onto the thin aqueous layer, whereby a supported bilayer displaying the affinity tag is produced; (c) providing a labeled ligand-chimera which also displays a ligand that binds to the affinity tag displayed on the supported bilayer; (d) contacting and binding the labeled ligand-chimera with the affinity tag displayed on the supported bilayer. The method can further comprise the step (e) contacting a live cell with the labeled ligand-chimera bound to the affinity tag displayed on the supported bilayer to observe cell-cell interactions.

The substrate of the assay system preferably comprises any material with a lipid-compatible surface such as SiO₂, MgF₂, CaF₂, mica, polydimethyl siloxane (PDMS), or dextran. SiO₂ is a particularly effective substrate material, and is readily available in the form of glass, quartz, fused silica, or oxidized silicon wafers. These surfaces can be readily created on a variety of substrates, and patterned using a wide range of micro- and nano-fabrication processes including: photolithography, micro-contact printing, electron beam lithography, scanning probe lithography and traditional material deposition and etching techniques.

In another embodiment, the substrate can be in an array format, having barrier materials to separate each corral/compartment in the array. Bilayer barrier materials can include polymers, such as photoresist, metals, such as chrome and gold, and minerals such as aluminum oxide. Alternatively, effective barriers between membrane corrals can be achieved by leaving portions of the substrate free of membrane. The resulting gap serves as a barrier that prevents diffusive mixing between separate corrals.

In a preferred embodiment, the supported bilayer of the assay system comprises a lipid bilayer wherein the primary ingredient is an egg-phosphatidylcholine (PC) membrane. In the absence of dopants, cells do not adhere to this membrane. Other suitable lipids that do not permit cell adhesion include pure phosphatidylcholine membranes such as dimyristoyl-phosphatidylcholine or dipalmitoylphosphatidylcholine. Another suitable primary lipid component is phosphatidylcthanolaminc (PE), which is also, in addition to PC, a primary component.

In one embodiment, the lipid composition in the supported lipid bilayer can comprise dopants to vary bilayer properties. Preferred dopant lipids are a negatively, positively or neutrally charged lipid. In one embodiment, the dopant lipid is the negatively charged lipid phosphatidylserine (PS). Other potential dopants can be dipalmitoylphosphatidic acid (PA), distearoylphosphatidylglycerol (PG), phosphatidylinositol, 1,2-dioleoyl-3-dimethylamonnium-propane, 1,2 dioleoyl-3-trimethylammonium-propane (DAP), dimethyldioctadecylammonium bromide (DDAB), 1,2-diolcoyl-sn-glycero-3-ethylphosphocholine (ethyl-PC), N-(7-nitrobenz-2-oxa-1,3-diazol-4-yl)-1,2-dihexadecanoyl-sn-glycero-3-phosphoethanolamine ammonium salt (NDB-PE). Suitable neutral lipid dopants include cerebrosides and ceramides. The amount of the dopant is selected based on the property of the dopant. For a lipid dopant, 2 to 10%, up to 20% is preferred.

In a preferred embodiment, the planar supported bilayers are formed by fusion of small unilamellar vesicles (SUV) with clean silica substrates according to the methods described in Salafsky, J., J. T. Groves, and S. G. Boxer, Architecture and function of membrane phospholipids in erythrocytes as factor in adherence to endothelial cells in proteins, Biochemistry, 1996, 35: 14773-14781, and U.S. Pat. No. 6,228,326, both of which are hereby incorporated in their entirety.

Generally, a lipid solution in chloroform is evaporated onto the walls of a round bottom flask that is then evacuated overnight. Lipids are resuspended in distilled water by vortexing moderately for several minutes. The lipid concentration at this point should be around 3 mg/ml. The lipid dispersion is then probe sonicated to clarity on ice, yielding small unilamellar vesicles (SUV). The SUVs were purified from other lipid structures by ultracentrifugation for 2 hours at 192,000 g. SUVs were stored at 4° C. and typically were stable for a few weeks to several months. The SUVs are fused onto the aqueous phase on the substrate. The vesicles spontaneously assemble in a matter of seconds to form a continuous single bilayer on the substrate. Excess vesicles can be rinsed away while maintaining the membrane bilayer under bulk aqueous solution at all times.

A planar supported bilayer is formed on the substrate with a thin aqueous layer between the bilayer and the substrate. In a preferred embodiment, the lipid bilayer displays a biological molecule, preferably an affinity tag having a known binding partner or having a known affinity molecule that can be attached. Referring now to FIG. 1B, in a preferred embodiment, the bilayer would be formed from biotinylated vesicles which thereby form a bilayer having biotin displayed, permitting the binding pair of streptavidin and biotin to be used. Other suitable affinity tags include polysaccharides, lectins, selecting, nucleic acids (both monomeric and oligomeric), proteins, enzymes, lipids, antibodies, and small molecules such as sugars, peptides, aptamers, drugs, and other ligands, and their binding partners.

In a preferred embodiment, ligands and biomolecules which one desires to be displayed by the supported bilayer are linked to the binding partner of the affinity tag, forming a ligand-chimera. The ligand-chimera is contacted and subsequently bound to the affinity tag displayed on the supported bilayer. For example, as shown in FIG. 1B, the labeled ligand-chimera is comprised of an epidermal growth factor (EGF) protein attached to streptavidin on one end and labeled with a detectable label on the other end. The EGF-Streptavidin chimera is contacted with the supported bilayer displaying biotin and the EGF-Streptavidin is captured and bound and thereby displayed.

In another embodiment, the ligand is a soluble signaling ligand. Examples of suitable soluble signaling ligands include peptides, proteins, membrane proteins, membrane-related proteins, receptors, antibodies, dyes, probes and other small molecules, polysaccharides, lectins, selectins, nucleic acids (both monomeric and oligomeric), proteins, enzymes, lipids, antibodies, and small molecules such as sugars, peptides, aptamers, drugs, and other soluble ligands such as other growth factors, cytokines, and hormones, tumor necrosis factors, G protein-coupled receptors (GPCRs), membrane-bound ligands, and cell-cell communication-related ligands such as cadherins, ephrins, etc.

In one embodiment, the ligand of the labeled ligand-chimera is an ephrin A1 (EA1) protein attached to an affinity tag with a known binding partner and a detectable label. In another embodiment, the ligand of the labeled ligand-chimera is a glycosylphosphatidyl inositol (GPI) anchored signaling ligand attached to both an affinity tag with a known binding partner and a detectable label. And in another embodiment, the ligand of the labeled ligand-chimera is a membrane-anchored signaling ligand attached to both an affinity tag with a known binding partner and a detectable label.

Methods of labeling molecules are well known to those of skill in the art. Preferred labels are those that are suitable for use in in situ hybridization or binding reactions. The ligand-chimera may be detectably labeled prior to the hybridization or binding reaction. Alternatively, a detectable label which binds to the hybridization product may be used. Such detectable labels include any material having a detectable physical or chemical property and have been well-developed in the field of immunoassays.

As used herein, a “label” is any composition detectable by spectroscopic, photochemical, biochemical, immunochemical, or chemical means. Useful labels in the present invention include radioactive labels (e.g., ³²P, ¹²⁵I, ¹⁴C, ³H, and ³⁵S), fluorescent dyes (e.g. fluorescein, rhodamine, Texas Red, etc.), electron-dense reagents (e.g. gold), enzymes (as commonly used in an ELISA), calorimetric labels (e.g. colloidal gold), magnetic labels (e.g. Dynabeads™), and the like. Examples of labels which are not directly detected but are detected through the use of directly detectable label include biotin and dioxigenin as well as haptens and proteins for which labeled antisera or monoclonal antibodies are available. The particular label used is not critical to the present invention, so long as it does not interfere with the in situ hybridization of the stain. In one embodiment, the detectable label is a fluorescent label. In a specific embodiment, Alexa Fluor 647, NBD and Hoechst 33342 are preferred for use with the supported bilayer assay system.

In another embodiment, the fluid SLBs are used for the presentation of soluble signaling ligands to cells in culture to promote cell adhesion. In one embodiment, it was found that membrane-tethered EGF is sufficient to promote cell adhesion and the fluidity of membrane-tethered ligands enhances its efficacy. Dynamic local enrichment of EGF molecules by reaction-diffusion processes was observed. The stretched morphology of the cells and the existence of focal adhesions suggest that the underlying substrate has been locally remodeled by ECM secretion. This process, however, is triggered by membrane displayed EGF. Through competition by inhibitory antibodies and EGFR kinase inhibitors, we demonstrated that this is an EGF-EGFR interaction-dependent phenotype and that kinase activation of the EGFR is also required. By studying the temporal adhesion of cells to EGF-SLB it is clear that full adhesion takes several hours, suggesting signaling through EGFR up-regulates a genetic program stimulating cell-adhesion.

This fluidity-based soluble ligand display system offers an experimental environment in which one can monitor dynamic reorganization and endocytosis of soluble ligands on a planar platform in the absence of ligands in solution. By eliminating ligands in solution, improved observation of soluble signaling molecules is possible because background fluorescence intensity is minimal in this system.

The ligand display strategy reported herein provides a new dimension to controlling soluble ligand exposure to cells in culture. Display of soluble signaling ligands in an array format allows for the utilization of developed membrane array technologies to present soluble ligands to cells in various configurations. This strategy will be useful in understanding the biology of ligand-receptor interactions as well as developing patterned soluble ligand-based high-throughput cell screening assays for medical diagnostic and cell biological applications. This system is expected to be applicable to other soluble ligands such as other growth factors, cytokines, and hormones as well as membrane-bound ligands (e.g., ephrins).

One objective of the present invention is the development of new, hybrid technologies that interface live cells with non-living materials. This involves deciphering the molecular language by which cells communicate, developing new methodologies for the manipulation and control of biological molecules, and the integration of these developments into functional systems. Thus, the invention relies on reassembly of lipids and proteins, purified from live cell membranes, into membrane structures supported on inorganic scaffolds. These supported membranes recapitulate many of the properties of live cell membranes. Most significantly, live cells can form functional signaling junctions with supported membranes. Hallmark examples of hybrid live cell-supported membrane junctions can be seen in the formation of immunological synapses between living cancer cells and supported membranes displaying the appropriate cognate ligands (FIG. 1). The supported membrane mimics the natural ephrin ligand presenting cell surface sufficiently well to trick the metastatic cancer cell into behaving as though it had engaged a living cell. The success of this strategy stems from the ability of reconstituted cell surface signaling and adhesion molecules in the supported membrane to diffuse freely and to engage their cognate receptors on the cancer cell in a life-like manner. Freedom of movement enables coalescence of proteins into signaling complexes and larger scale spatial patterns. Therefore, in one embodiment, the described supported membrane-based methods provide a uniquely powerful solution to the growing demand for cellular diagnostic tools and clinical applications.

In another aspect, development of sophisticated diagnostic technologies to tailor appropriate combinations of therapeutics to individual patients is of paramount importance for the future eradication of cancer. Current techniques for genetic screening and protein expression profiling, while fortuitously successful in some cases, are indirect and cannot be expected to comprehensively cover the disease space. There will be a need for high-information-content live-cell screens suitable for analysis of cells from individual biopsies as a general requirement for broadly successful personalized cancer treatment. Therefore, it is contemplated that assay systems and methods such as described in Example 3 will provide a uniquely powerful solution to the growing demand for such cellular diagnostic tools.

### Example 1

Epidermal growth factor (EGF) and the EGF-receptor tyrosine kinase (EGFR) were chosen as a prototypic signaling system to evaluate the SLB platform. EGFR is a member of the type-I (ErbB) receptor tyrosine kinases (RTKs) and is activated by a number of ligands from the EGF family. This results in receptor dimerization and a cascade of signaling events culminating in a number of biologic end points including proliferation. ErbB de-regulation is a common event in human cancer where EGFR and a second family member, ErbB2, have become targets for directed therapeutic interventions such as Tarceva™, Herceptin™ and Iressa™. It is clear that molecular understanding of EGFR and ErbB2 has a translational impact, and a more detailed understanding of the molecular interactions of these molecules may yield further clinical benefit. Recent insights into the molecular mechanisms of EGFR signaling suggest that localization of EGFR on the cell membrane enhances receptor dimerization/clustering which is pre-requisite for ligand binding and activation of receptor kinase activity. See A. Sawano, S. Takayama, M. Matsuda, A. Myyawaki, Dev. Cell 20023, 245 and J. Ichinose, M. Murata, T. Yanagida, Y. Sako, Biochem. Biophys. Res. Commun. 2004, 324, 1143. Applying the fluid membrane-tethered ligand display method reported herein to the EGF-EGFR system has clear benefits. The system allows for fast local enrichment of EGF induced by the EGF-EGFR interactions, facile in situ monitoring of fluorescently-labeled EGF and temporal analysis of cellular phenotypes in a surface assay format.

The design of an EGF-modified fluid SLB (EGF-SLB) assay is outlined in FIG. 1B. To measure the fluidity of lipid bilayers (DMOPC, 1,2-dimyristoleoyl-sn-glycero-3-phosphocholine) with and without substrate-bound EGF, a focal region of the membrane was photobleached, and fluorescence from NBD (lipids alone) or Alexa Fluor 647 (EGF-modified lipids) was monitored. Photobleached regions for both bare lipids (green) and EGF-modified lipids (red) recovered fluorescence indicating they are fluid (FIG. 1B, inset). Interestingly, the EGF-modified lipids were slightly less fluid than the bare NBD-modified lipids, suggesting EGF binding to the SLB alters the fluidity of the EGF-tethered lipids.

As a practical test of this system, the EGF-EGFR interactions between the EGF-SLB and live cells were examined. The immortal, non-transformed breast epithelial cell line, MCF10a, was chosen for this purpose as these cells express EGFR and are dependent on EGF signaling for proliferation and survival (all the cells in this paper refer to MCF-10a cells). MCF-10a cells in serum-free, growth factor free DMEM/F-12 media (˜300,000 cells per ml) were applied to an EGF-SLB array and to a streptavidin-modified lipid membrane without EGF molecules. The cells were incubated at 37° C. for 20 hrs after which they were gently washed with DMEM/F-12 media and visualized by epifluorescence microscopy (TE300, Nikon, Inc.). Analysis of membranes post-washing revealed attachment of cells to the EGF-SLB array but not to the streptavidin-modified lipid membrane (FIG. 2) suggesting EGF-dependent attachment of cells to the lipid surface. However, it was unclear whether direct ligand-receptor interaction alone was responsible for cell-membrane attachment, or whether EGFR signaling modulated cell attachment to the EGF-SLB via secondary mechanisms. To investigate whether the direct binding of EGF to EGFR facilitated attachment a competing antibody for EGFR (mAb225) was added to the cells. The presence of 3 ng/mL competing antibody reduced the number of cells attached to the membrane by 94% after 20 hrs (FIG. 2, bottom left panel). This confirmed the specificity of the EGF-EGFR interactions and that it is required for cell-to-EGF-SLB attachment. EGF stimulation of EGFR kinase activity signaling activates a number of downstream pathways, some of which regulate cytoskeletal molecules, cell attachment and motility. Therefore, we next tested if EGFR kinase activity is required for attachment by treating cells with Tarceva™, a specific kinase inhibitor of EGFR. When the assay was performed in the presence of Tarceva™, there was a significant reduction in the number of cells attached to the membrane (FIG. 2, bottom right panel) confirming that activation of EGFR kinase activity is required for cell attachment.

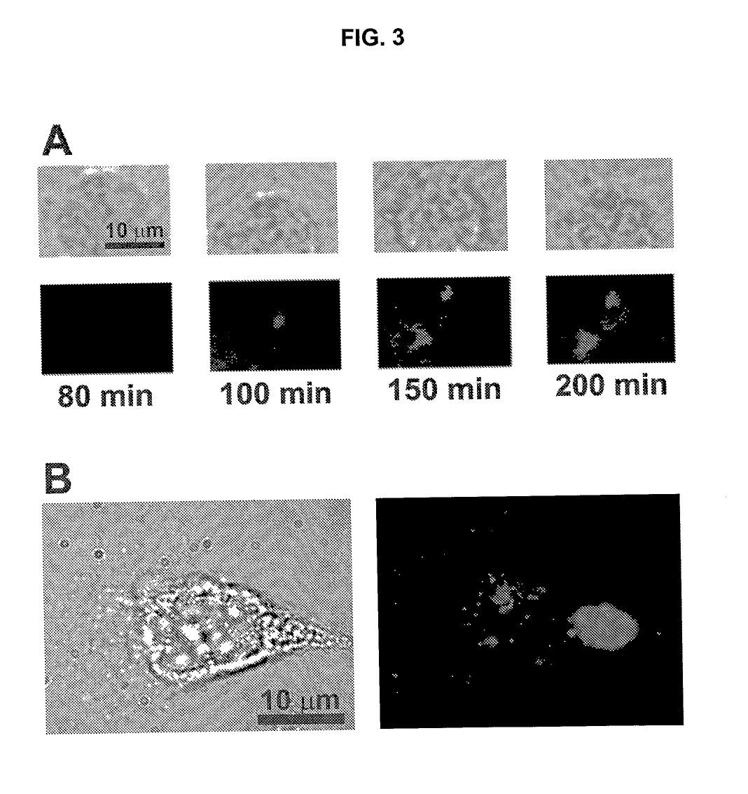

To understand the temporal and spatial kinetics of the EGF-EGFR interaction, time-lapse experiments were employed to observe cell attachment to the EGF-SLB and subsequent EGF localization. This dynamic interaction was monitored using bright field microscopy to image cells and epifluorescence microscopy to image the EGF-coupled Alexa Fluor 647 (FIG. 3). Cells were observed to weakly adhere to the surface as early as 80 min post plating. At this time, EGF was still randomly distributed across the surface. By 100 min, EGF molecules were observed to cluster into small focal points, which increased in size in a temporal fashion. These small clusters began to form larger clusters at around 150 min (FIG. 3A). After 20 hr, a cell is spreading and adhered to the surface with many distinctive EGF clusters (FIG. 3B). These clusters are reminiscent of focal adhesions required for cell-attachment to substratum. Since these EGF clusters appear to lie partially out of the supported membrane plane, as determined by focusing the microscope at different positions, we suspect that these clusters could be endocytosed EGFRs with bound EGFs and fluorophore labels. Since natural trigger of EGFR by EGF is followed by endocytosis, we interpret this observation as higher support of signaling functionality of membrane-tethered EGF. It should also be noted that cells cannot apply tensile forces to membrane adhesion sites; the fluid membrane will simply flow under such forces. The stretched cell attachment phenotype (FIGS. 2 and 3B) clearly indicates the presence of tensile forces, suggesting that the cells are anchored to the underlying solid substrate through focal adhesion sites. Formation of these focal adhesions likely involves remodeling of the surface by secretion of ECM proteins.

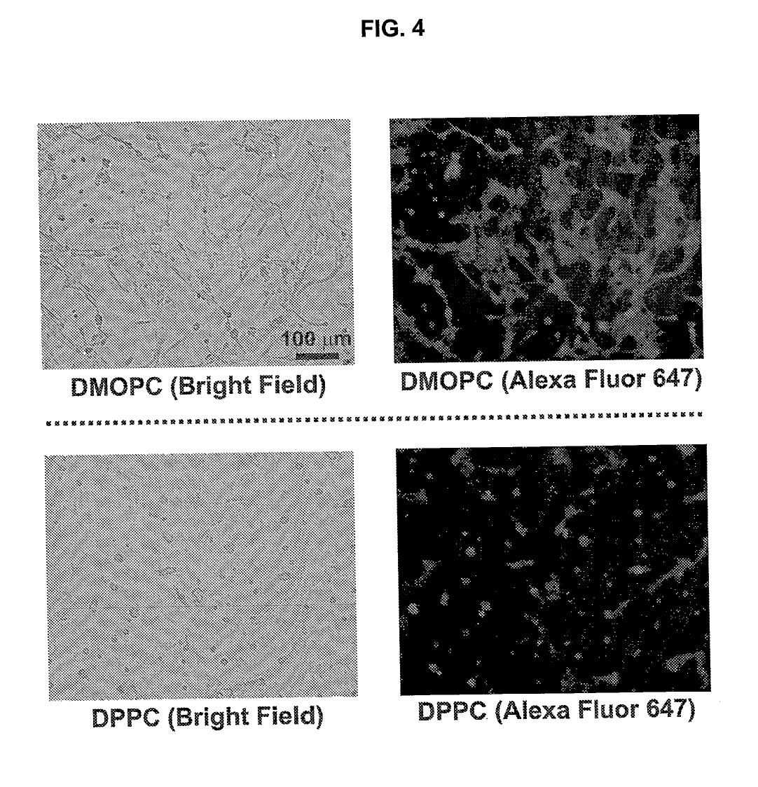

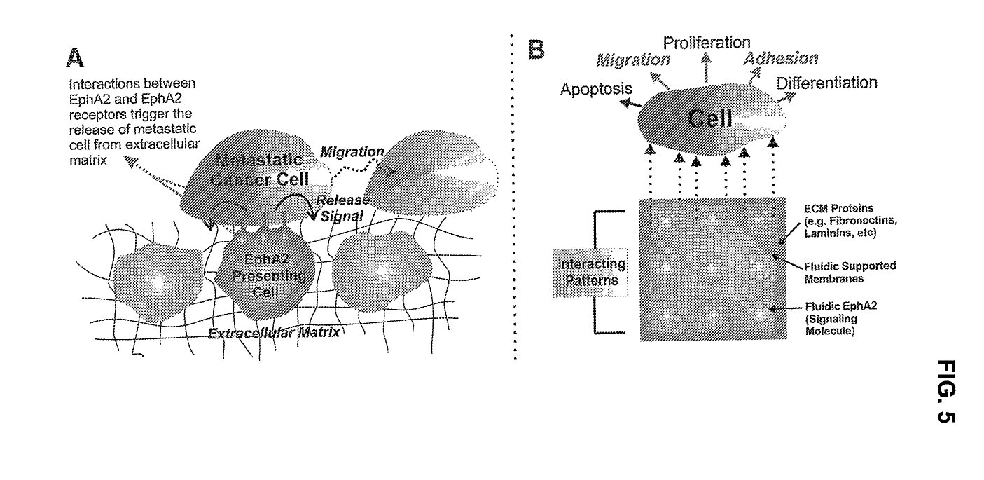

Clustering of EGFR on the cell surface is a pre-requisite for signal activation by ligand binding and is dependent on ligand diffusion across the SLB. Therefore it was hypothesized that fluidity of membrane-tethered EGF would facilitate this process. To test this hypothesis, direct comparison of the DMOPC-based system was made to the DPPC (1,2-dipalmitoyl-sn-glycero-3-phosphocholine)-based system as DMOPC is much more fluid than DPPC (at 37° C., the diffusion constant for DMOPC is 9 μm²/sec and the diffusion constant for DPPC is 0.1 μm²/sec; Avanti Polar Lipids, Inc., Alabaster, Ala.). M. B. Forstner, C. K. Yee, A. Parikh, J. T. Groves, Sparse Protein Binding Alters Long-Range Lipid Mobility via Modulation of Phase Transition Behavior in Membranes, in preparation; J.-F. Tocanne, L. Dupou-Cezanne, A. Lopez, Prog. Lipid Res. 1994, 33, 203. Cells were applied to DMOPC- or DPPC-based EGF-SLB using the procedure described below, and EGF localization and cell attachment were observed after 20 hrs by bright field and epifluorescence microscopy. Significantly more cells adhered to the more fluid, DMOPC-based membrane substrate than to the less fluid, DPPC-based membrane substrate (˜3.7-fold more live cells were adhered to DMOPC-based membrane arrays, FIG. 4). Cells adhered to the DMOPC-EGF-SLB exhibited increased cell spreading, indicative of a motile phenotype, compared to the DPPC-EGF-SLB. Cells attached to the DMOPC-based EGF-SLB surface displayed EGF clusters at the location where the cells were adhered. In contrast, fewer EGF clusters were found where cells were attached to the DPPC membrane arrays (FIG. 4). These results suggest that supported membrane fluidity facilitates localized clustering of EGF, which is essential for its signaling functionality.

Experimental Section. EGF-Modified SLB was fabricated with the following procedures. First, biotinylated lipid vesicles along with NBD-modified vesicles have been prepared using existing methods described in E. Sackmann, M. Tanaka, Trends Biotechnol. 2000, 18, 58; J. T. Groves, Angew. Chem. Int. Ed. 2005, 44, 3524; C. K. Yee, M. L. Amweg, A. N. Parikh, J. Am. Chem. Soc. 2004, 126, 13962; and L. Kam, S. G. Boxer, Langmuir 2003, 19, 1624, and hereby incorporated by reference for all purposes. In short, the desired lipids were dissolved in chloroform, and then the chloroform was evaporated using a rotary evaporator. The lipids were thoroughly dried under nitrogen gas and then hydrated with 1 mL of water. The hydrated lipids were extruded through 100 nm-sized pore filters and stored at 4° C. until the day of the experiments. Then, the vesicles (3% biotin-modified DPPE, 2% NBD-modified PC, and 95% DMOPC purchased from Avanti Polar Lipids, Inc., Alabaster, Ala.) were allowed to warm to room temperature. Next they were ruptured on a piranha-etched microscopic cover glass (Fisher Scientific, Pittsburgh, Pa.) in 25 mM NaCl solution. The resulting lipid-bilayered glass substrate, immersed in NaCl solution, was sealed in an Attofluor cell chamber (Invitrogen Corp., Carlsbad, Calif.). Subsequently, EGF molecules conjugated to streptavidin and Alexa Fluor 647 (150 μl at 100 μg/ml) were applied to the biotinylated membrane-modified glass substrate for 45 min at room temperature (approximately one biotinylated EGF molecule was bound to each streptavidin-modified Alexa Fluor 647, leaving three binding sites for each streptavidin to bind to a membrane-bound biotin molecule; Invitrogen Corp., Carlsbad, Calif.). This allowed attachment of EGF molecules to the membrane via streptavidin-biotin interactions. The NaCl salt solution immersing the SLB was then exchanged by washing the Attofluor cell chamber three times with DMEM/F-12 media (GIBCO, Invitrogen Corp., Carlsbad, Calif.). This washing step served the dual purpose of removing unbound EGF-streptavidin-Alexa Fluor 647 molecules and immersing the SLB in media that was suitable for the desired cells to survive, while still retaining membrane fluidity. At this point 1 mL of MCF-10a cells (3×10⁵ cells/mL) was added to the Attofluor cell chamber. The chamber was then wrapped in parafilm, with holes to allow oxygen into the chamber, and the cells were incubated at 37° C. for 20 hours. After the incubation period, the Attofluor cell chamber was washed three times with DMEM/F-12 media to remove any non-adhered MCF-10a cells. The cells were then imaged using bright field and epifluorescence microscopy.

FRAP experiments were conducted to verify the fluidity of the phospholipids in the bilayer, labeled with 2% NBD, and the lipid-tethered EGF-streptavidin complex, labeled with Alexa Fluor 647, on a glass substrate. First, both fluorophores were photobleached over the span of approximately 3 min. The photobleached area (the dark octagon in the center of the images) was then allowed to recover for 10 min, and epifluorescence images were taken (the bilayer was exposed to the excitation wavelengths for 3 seq). The resulting images were then false-colored and processed using Adobe Photoshop 7.0 (green for NBD and red for Alexa Fluor 647). Recovery of fluorescence for both NBD and Alexa Fluor 647 confirms that the DMOPC phospholipids in the bilayer, as well as the EGF bound to the streptavidin, were fluid under the experimental conditions.

For the studies using DPPC, the initial lipid concentrations of the vesicles were 3% biotin-modified DPPE, 2% NBD-modified PC, and 95% DPPC. After extruding through 100 nm-sized pore filters, the vesicles were extruded through 30-nm-sized pore filters so they would be smaller and easier to rupture. Before rupturing the vesicles, they were heated to 50° C., as was the spreading solution and the NaCl salt solution. The piranha-etched microscopic cover glass was also heated above 50° C. All of these heating steps were required to ensure the lipids were in the fluid phase while the bilayer was being formed. All other steps remained the same as when using DMOPC.

A human breast epithelial cell line, MCF-10a, was cultured in scrum-rich media consisting of DMEM/F-12 media (GIBCO, Invitrogen Corp., Carlsbad, Calif.), hydrocortisone (500 ng/mL), horse serum (5% vol/vol), bovine insulin (0.01 mg/mL), and EGF (20 ng/mL). The day of the experiments, they were treated with trypsin-EDTA, washed twice with 1×PBS, centrifuged, and 3×10⁵ of the cells were re-suspended in 1 mL for each experiment. These 1 mL aliquots were then incubated in a 37° C. water bath until they were added to the EGF-SLB. For the studies with Tarceva™ and mAb225, the cells were incubated with either Tarceva™ or mAb225 for 45 minutes in a 37° C. water bath before being added to the EGF-SLB. All other steps were as before.

For the studies to count cells adhered to EGF-SLBs, the initial lipid concentrations were as before, but with an additional 2% of the primary lipid constituent substituted for 2% NBD-PC (3% biotin-modified DPPE and 97% DMOPC or DPPC). After the 20-hour incubation of the cells on the EGF-SLBs, the chamber was washed three times with DMEM/F-12 media as before, to remove non-adhered cells. Then the cells were stained with Hoechst 33342 (100 μl at 1 μg/ml) for 10 minutes and the chamber was washed four more times with DMEM/F-12 media to remove any unbound Hoechst 33342. Then the cells were imaged using bright field and epifluorescence microscopy.

A TE300 Nikon inverted microscope with a mercury arc lamp was used for epifluorescence illumination and a 100 W halogen lamp for bright field illumination. FIG. 3A was taken with a Hamamatsu Orca CCD camera (Hamamatsu Corp., Hamamatsu City, Japan) and FIGS. 2, 3B, and 4 were taken with a CoolSnap HQ CCD camera (Roper Scientific, Inc., Tucson, Ariz.). SimplePCI (Compix, Inc. Imaging Systems, Cranberry Township, Pa.) and MetaMorph (Molecular Devices Corp., Downington, Pa.) software was used to collect and analyze the images, which were then further processed using Adobe Photoshop 7.0. Alexa Fluor 647 was imaged using a Cy5 filter cube and NBD was imaged using an NBD/HPTS filter cube. For the cell counting studies Hoechst 33342 was imaged using a DAPI/Hocchst/AMCA filter cube. All filter cubes were purchased from Chroma Technology Corp., Rockingham, Vt.

### Example 2

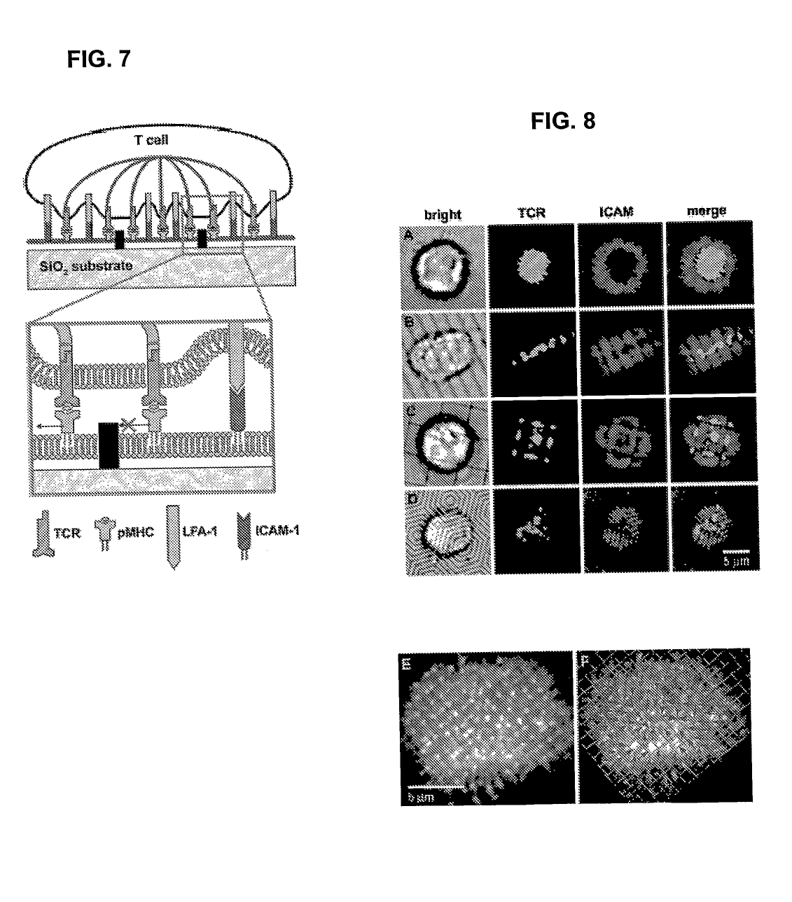

An experimental platform was developed that enables direct manipulation of IS patterns in living T cells. A supported membrane, consisting of a continuous and fluid lipid bilayer coating a silica substrate (E. Sackmann, Science 271, 43 (1996)), is used to create an artificial APC surface (J. T. Groves, M. L. Dustin, J. Immunol. Methods 278, 19 (2003)). Inclusion of glycosylphosphatidylinositol (GPI)-linked pMHC and ICAM-1 into the supported membrane is sufficient to enable IS formation between a T cell and the synthetic surface. This hybrid live cell-synthetic bilayer IS is illustrated schematically in FIG. 7. Fluidity is a characteristic property of supported bilayers and distinguishes them from solid and polymeric substrates. Movement within the bilayer, however, can be manipulated by fabricating geometrically defined patterns of solidstate structures on the substrate (FIG. 7) (J. T. Groves, N. Ulman, S. G. Boxer, Science 275, 651 (1997)). It was posited that such substrate-imposed constraints might be used to guide molecular motion in the supported bilayer and linked cell-surface receptors to generate alternatively patterned synapses.

Silica substrates displaying various configurations of chromium lines (100 nm wide and 5 nm high) were fabricated using electron-beam lithography (B. L. Jackson, J. T. Groves, J. Am. Chem. Soc. 126, 13878 (2004)). Supported proteolipid membranes were assembled on these substrates by vesicle fusion. As receptors on the T cell surface patterns, which create an array of isolated membrane corrals (FIG. 8C). More elaborate constraint designs, such as a mosaic of concentric hexagonal barriers (FIG. 8D), were also used. A diverse collection of spatially mutated IS patterns were generated to investigate the effects of spatial constraints on synaptic signaling.

The chromium barriers also enabled us to provide insight into basic mechanisms of IS formation. For example, a 1-mm grid caused fragmentation of the IS into more than 100 microsynaptic TCR clusters that were stable for more than 30 min (FIG. 8E) despite the rapid TCR-pMHC off rate (˜0.06 s⁻¹). Because TCR motion can only be constrained by the grid through engagement with pMHC, the stability of corralled TCR microclusters indicates that the TCRs in each microcluster move collectively as a multimeric unit. Otherwise, individual TCRs would percolate over the barriers during disengagements from pMHC, and the stable trapping of microclusters would not be observed. The position of each TCR-PMHC microcluster within its corral revealed the direction of transport and could be used to compile a transport map of the IS (FIG. 8F). The microclusters on grids were generally “pulled” to the corner of the corral nearest the center of the IS, and images could be quantified to reveal the high degree of centralized TCR organization in frustrated synapses (data not shown). Typically, one TCR-pMHC cluster is observed per corral for the 1-, 2-, and 5-mmsquare grids that were studied, suggesting that TCR clustering occurred only after pMHC engagement. Thus, if TCR were substantially preclustered, one would expect a stochastic distribution of microclusters within the corrals rather than the even distributions we observed on the 1-mm and 2-mm grids. Collectively, this set of observations supports a three-step process by which the mature IS is formed: (i) TCR engagement of pMHC, (ii) TCR-pMHC assembly into microclusters, and (iii) directed transport of microclusters to form the c-SMAC.

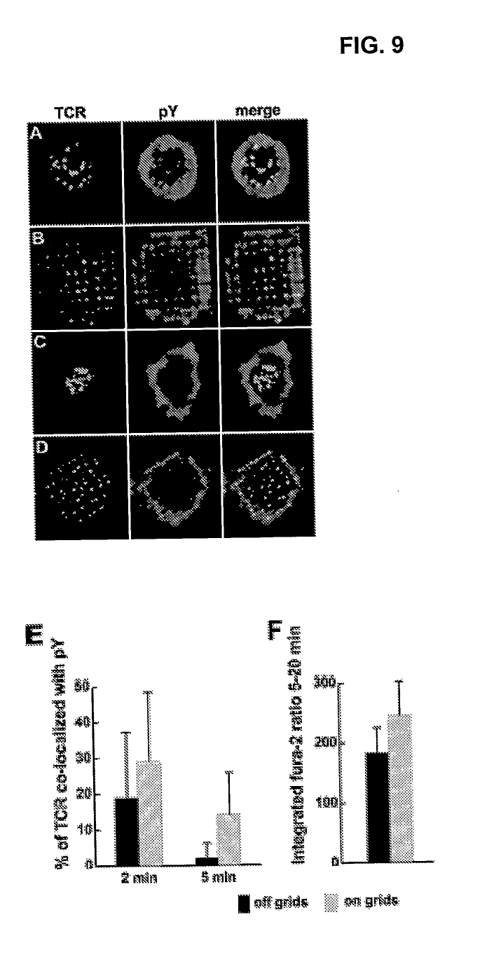

Using cytoplasmic distribution of phosphorylated tyrosine (pY) residues associated with TCR clusters, signaling activity specific to each TCR cluster within constrained synapse motifs was next measured (K. H. Lee et al., Science 295, 1539 (2002)). At early time points, pY patterns were similar in both native and repatterned synapses (FIGS. 9, A and B). However, at 5 min, TCR clusters in the natively pattered IS were observed only in the c-SMAC region and had very low pY levels (FIG. 9C). In contrast, TCR clusters that had been stably restrained to the periphery of the contact area by the substrate grids retained high specific pY levels (FIG. 9D). This effect was restricted to the periphery, because TCR clusters trapped in more central regions of spatially modified synapses lost their pY signal in a time frame similar to those observed in native synapses. The duration of TCR-pY signaling thus correlated with radial position of the TCR rather than with cluster size. Overall, the extent of specific pY associated with TCR clusters above the local background was significantly greater in the IS that had been spatially constrained by the grid (FIG. 9E).

Another key measure of signaling activity is the flux of intracellular Ca²⁺ induced by TCR antigen recognition, which integrates the outputs of all TCR signaling events in the IS (D. J. Irvine, M. A. Purbhoo, M. Krogsgaard, M. M. Davis, Nature 419, 845 (2002)). The integrated Ca²⁺ response was significantly higher in cells with spatially constrained IS as compared with those with native synapses (FIG. 9F). Thus, mechanical trapping of TCR in the IS periphery augments early TCR-associated pY levels, as well as the elevation of cytoplasmic Ca²⁺.

These experiments provide insight into how signaling is extinguished in individual TCR clusters in the IS, which may be attributed to temporal or spatial processes such as recruitment of inhibitors or changes in the actin cytoskeleton that feed back on signaling. The hybrid live cell-supported membrane platform made it possible to physically impede receptor translocation to prevent c-SMAC formation, allowing the determination that radial location represents a critical parameter in the IS. In physiological terms, it is possible that some APCs may use their own cytoskeletons to restrict transport of pMHC or costimulatory molecules in a related manner. Impeding TCR cluster translocation to the c-SMAC might thus represent a means of augmenting T cell activation (S. Y. Tseng, M. Lu, M. L. Dustin, J. Immunol., in press; M. M. Al-Alwan, G. Rowden, T. D. Lee, K. A. West, J. Immunol. 166, 1452 (2001)). Potentially, the ability to induce spatial modifications in model cell-cell interfaces could be useful in exploring spatial organization of membrane domains and proteins on the cell surface, receptor signaling activity, and cytoskeletal regulation processes.

### Example 3

Many aspects of cancer result from aberrant signal transduction at the cell surface. Metastasis is one of the most deadly processes of cancer, and each of its phases (detachment, migration, invasion, growth, and survival) is regulated by cell-cell contact interactions and the associated signaling systems. For example, recent studies have found the EphA2 receptor tyrosinc kinase (RTK) to be frequently over expressed and functionally altered in aggressive tumor cells (40% of breast cancers [B. L. Jackson, J. T. Groves, J. Am. Chem. Soc. 126, 13878 (2004)]), and that these changes promote metastatic character (FIG. 2A) [M. M. Davis et al., Annu. Rev. Biochem. 72, 717 (2003)]. EphA2 is one of the Eph receptors, which constitute the largest family of RTKs and, together with their membrane-bound ephrin ligands, regulate a broad range of signaling processes at intercellular junctions. In addition to metastasis, Eph receptors are involved in oncogenic transformation and tumor-driven induction of angiogenesis. Since both the Eph receptors and their ephrin ligands are associated with the cell membrane, this family of cell surface signaling molecules are ideally suited to reconstitution into the hybrid live cell-supported membrane configuration.

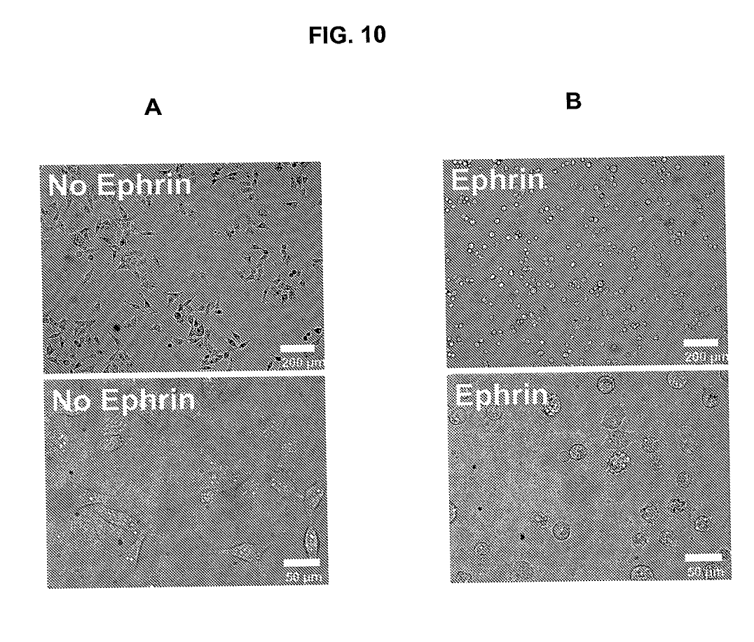

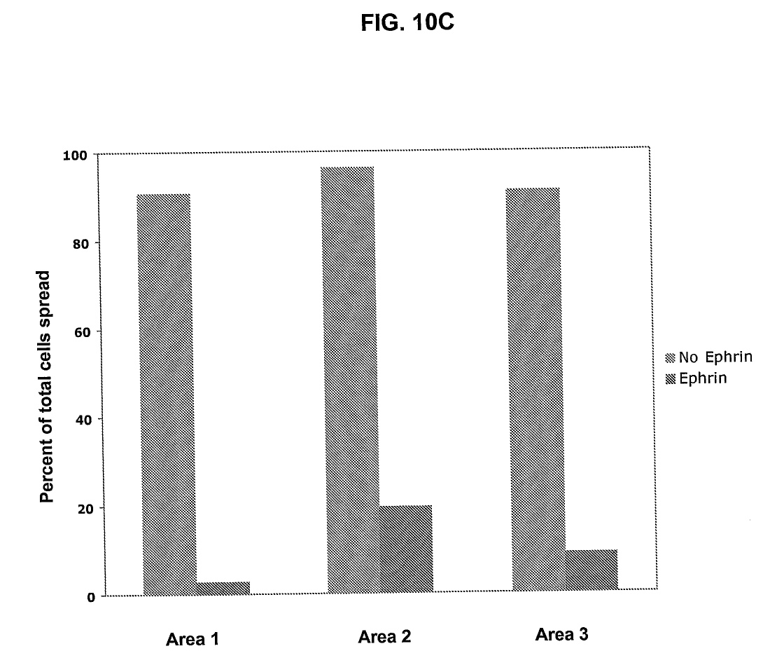

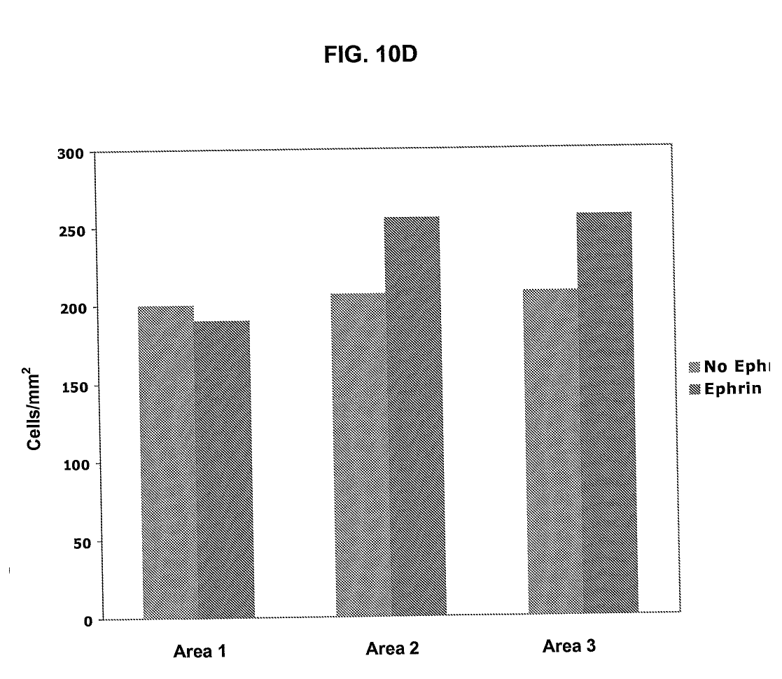

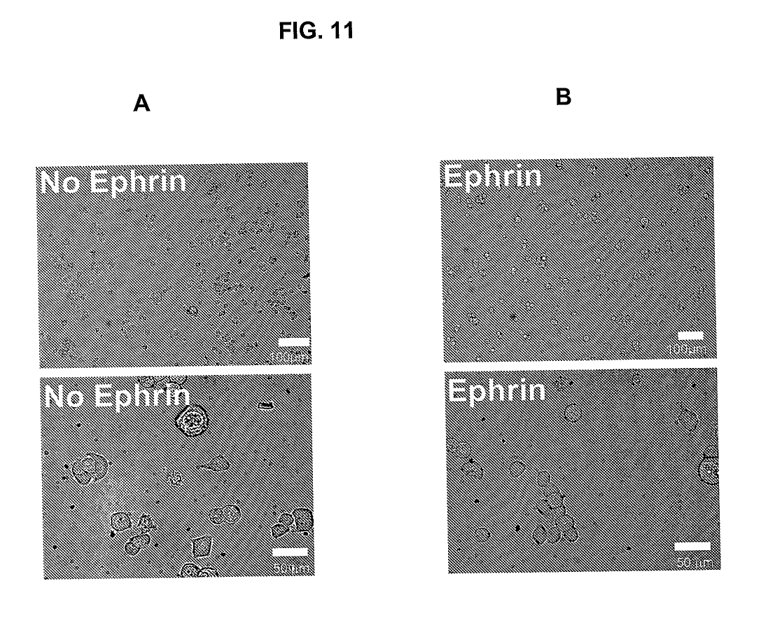

To test the ability of the SLB platform to distinguish between metastatic and non-metastatic cells, an ephrin A1-functionalized supported lipid bilayer (EA1-SLB) was designed. This environment was then presented to various cancer cell lines. Decreased spreading was observed when metastatic cancer cells (MDAMB231) displaying the EphA2 receptor were cultured in this environment (FIG. 10C). When non-metastatic cancer cells (T47D) not displaying the EphA2 receptor were cultured under the same conditions, no change in behavior was observed (FIG. 11).

The benefits of successfully engineering a supported membrane to engage and communicate with cancer cells are multifold. From a research perspective, the exquisite chemical control provided by supported membranes offers an invaluable tool for the elucidation of fundamental signaling mechanisms. Better understanding of these processes in cancer is sure to lead to new modalities for therapeutic intervention. The most direct impact on cancer survival rates, however, may well be realized by utilizing the system as a cellular diagnostic. It is contemplated that a mosaic of supported membranes is made that display the various cell surface signals encountered in normal tissue. Biopsy cells from an individual patient would then be cultured on this artificial cell surface, and their behavior under the influence of various drugs would be examined. Key to this strategy is the ability to functionally reconstitute the appropriate cell surface signals so that critical behaviors, such as invasion, are accurately revealed. The remarkable successes of supported membranes in capturing subtleties of T cell recognition in Example 2 demonstrates that this system can be implemented successfully as described herein. Furthermore, others have shown that different environments such as 3-D cell culture systems drive cells to behave in completely different ways comparing to typical 2-D cell culture environments. This becomes critical when one needs to replicate in vivo experimental results on a bench top.

In another embodiment, the described supported membrane-based technologies can also be used to present patterns and functional molecules in ways that nature presents them to cells in vivo because supported membrane represents cell surface, and modified functional molecules are fluidic within supported membrane structures.

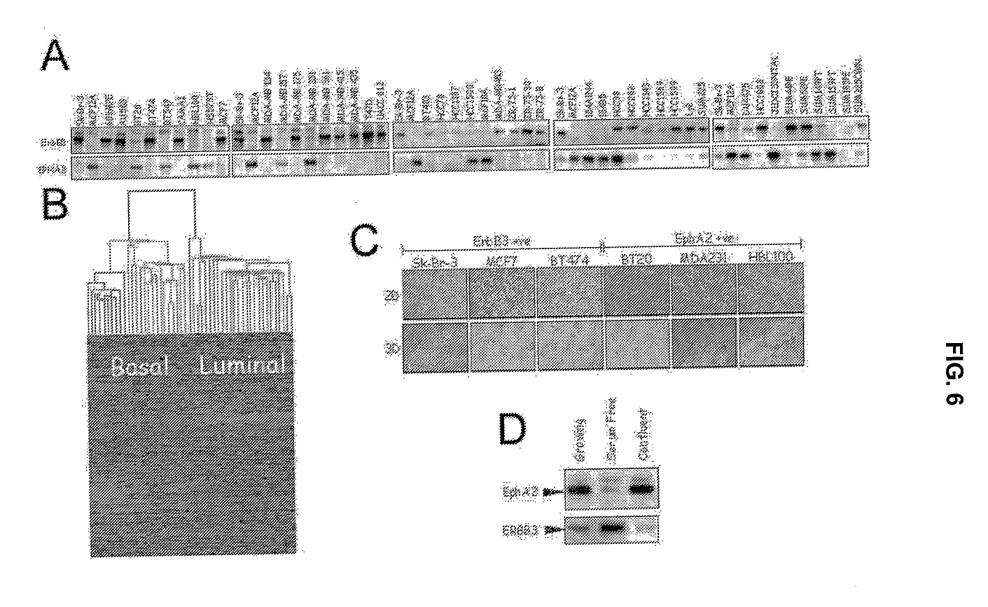

Hybrid live cell-supported membrane systems for cancer cell analysis will initially be constructed by incorporating ephrin ligands and related cell adhesion molecules, such as E-cadherin, into supported membranes (FIG. 5B). These molecules are generally associated with negative regulation of cell growth and migration at cell-cell contacts. Their successful reconstitution into supported membranes will enable the patterning of spatially defined signals onto the surface, which will govern the behavior of live cells. (FIG. 5B) Comparative observations of healthy and diseased cells within these patterned environments will be used to develop a comprehensive series of functional assays for cellular analysis.

In order to establish the validity of this strategy, the system will be comprehensively analyzed on a collection of more than 60 human breast cancer cell lines. Protein expression profiling indicates significant diversity within the collection (FIG. 6). At the same time, important reciprocal correlations between EphA2 and ErbB3 exist. Apparently, signaling through the ErbB and EphA2 pathways creates a homeostatic mechanism controlling proliferation and invasion. Multiple ways in which these critical pathways can become deregulated in cancer are represented within the collection of cell lines.

Once a basis set of behavior responses to specific supported membrane-displayed signals are established, the next phase of development will explore drug effects on these behavior response spectra. We will seek to identify and refine signatures of efficacy, which could be used as predictive markers for therapeutic value. These can then be exported as a set of live cell assays for cancer drug discovery to pharmaceutical groups. Our own core research efforts will emphasize miniaturization of the assays for diagnostic applications on patient biopsy samples.

The ultimate goal of this project is to create a suite of hybrid live cell-supported membrane assays that comprehensively reconstitute numerous functional aspects of cancer. Interactions between live cells from the patient with cell surface signals displayed on the supported membrane will create a thoroughly personalized assay, with which the full complement of potential therapeutic agents can be characterized (FIG. 5B). This type of micro-high throughput live cell assay will form an integral part of a comprehensive diagnostic process, which would also involve extensive genetic and protein expression profiling.

In constructing a fluidic membrane-based single cancer cell diagnosis system, there are five basic steps: (1) Fabrications of various functional fluidic substrates that present various compositions, shapes, density, and positions' of functional molecules that interact with cells, (2) Subsequent interactions between cells and membrane-based functional substrates, (3) Observe the adhesion, migration premium signal for metastasis), and proliferation of breast cancer cells based on different cellular environments (e.g., patterns) when compared to normal cells, (4) Based on what is learned from these studies, record and quantify specific cellular behaviors for single cell-based breast cancer diagnostics (for example, metastasis), and (5) Build massively arrayed single cell observation chambers based on microfluidics (e.g., multiplexed membrane-based cancer diagnostic chip).

The present examples, methods, procedures, specific compounds and molecules are meant to exemplify and illustrate the invention and should in no way be seen as limiting the scope of the invention. Any patents, publications, publicly available sequences mentioned in this specification are indicative of levels of those skilled in the art to which the invention pertains and are hereby incorporated by reference to the same extent as if each was specifically and individually incorporated by reference.

What is claimed is:

1. A ligand-modified fluid supported lipid bilayer (SLB) assay system to functionally display soluble ligands to cells in situ, the SLB assay system comprising a substrate supporting a membrane bilayer having an aqueous layer between the substrate and the bilayer, wherein a soluble signaling ligand is displayed by the membrane bilayer thereby permitting a cell to interact with the signaling ligand.

2. The SLB assay system of claim 1, wherein a thin aqueous layer is between the bilayer and the substrate.

3. The SLB assay system of claim 1, wherein the lipid bilayer displays a biological molecule, wherein the biological molecule is an affinity tag having a known binding partner or having a known affinity molecule that can be attached.

4. The SLB assay system of claim 3, wherein the biological molecule displayed by the lipid bilayer is biotin, thereby permitting a binding pair of streptavidin and biotin to be used.

5. The SLB assay system of claim 3, wherein the biological molecule displayed by the lipid bilayer is a suitable affinity tag selected from the group consisting of: polysaccharides, lectins, selecting, nucleic acids (both monomeric and oligomeric), proteins, enzymes, lipids, antibodies, and small molecules such as sugars, peptides, aptamers, drugs, and other ligands, and thereby forming a bilayer displaying the affinity tag.

6. The SLB assay system of claim 3, wherein a labeled ligand-chimera is captured by the affinity tag and thereby displayed by the lipid bilayer.

7. The SLB assay system of claim 6, wherein the labeled ligand-chimera is an epidermal growth factor (EGF) protein attached to streptavidin and a detectable label.

8. The SLB assay system of claim 6, wherein the ligand of the labeled ligand-chimera is a soluble signaling ligand attached to the binding pair of the displayed biological molecule and a detectable label.

9. The SLB assay system of claim 6, wherein the detectable label is a fluorescent molecule.

10. The SLB assay system of claim 6, wherein the ligand of the labeled ligand-chimera is an ephrin A1 (EA1) protein attached to an affinity tag with a known binding partner and a detectable label.

11. The SLB assay system of claim 6, wherein the ligand of the labeled ligand-chimera is a glycosylphosphatidyl inositol (GPI) anchored signaling ligand attached to both an affinity tag with a known binding partner and a detectable label.

12. The SLB assay system of claim 6, wherein the ligand of the labeled ligand-chimera is a membrane-anchored signaling ligand attached to both an affinity tag with a known binding partner and a detectable label.

13. A method of making an assay system comprising the steps of: (a) providing a substrate having a thin aqueous layer; (b) condensing a vesicle displaying an affinity tag by vesicle fusion process onto the thin aqueous layer, whereby a supported bilayer displaying the affinity tag is produced; (c) providing a labeled ligand-chimera which also displays a ligand that binds to the affinity tag displayed on the supported bilayer; (d) contacting and binding the labeled ligand-chimera with the affinity tag displayed on the supported bilayer.

14. The method of claim 13 further comprising a step (e) contacting a live cell with the labeled ligand-chimera bound to the affinity tag displayed on the supported bilayer to observe cell-cell interactions.
